(19) United States
(12) Patent Application Publication
(10) Pub. No.: US 2022/0054487 A1
(43) Pub. Date: Feb. 24, 2022

(54) METHODS FOR TREATING HETEROTOPIC OSSIFICATION

(71) Applicant: Clementia Pharmaceuticals Inc., Montreal (CA)

(72) Inventors: Clarissa DESJARDINS, Westmount (CA); Donna Roy GROGAN, Boston, MA (US); Jeffrey Neal PACKMAN, Weston, MA (US); Mark HARNETT, Stow, MA (US)

(21) Appl. No.: 17/413,227

(22) PCT Filed: Dec. 13, 2019
(86) PCT No.: PCT/CA2019/051803

§ 371 Date: Jun. 11, 2021

(30) Foreign Application Priority Data
Dec. 13, 2018 (CA) PCT/CA2018/051595

(51) Int. Cl. A61K 31/506; A61K 31/415; G16H 20/10; A61P 19/08

(52) U.S. Cl. CPC A61K 31/506; A61P 19/08; G16H 20/10; A61K 31/415

(57) ABSTRACT

The invention describes dosing regimens for treating subjects with fibrodysplasia ossificans progressiva characterized by a quiescent period and a non-quiescent period, wherein a therapeutically effective amount of palovarotene is administered to the subjects during the non-quiescent period, little or no palovarotene is administered to the subjects during the quiescent period, and imatinib is administered to the subjects during the quiescent period and optionally during the non-quiescent period. The dosing regimens can reduce heterotopic ossification, reduce the number of flare-ups, and/or reduce the severity of flare-ups in subjects suffering from fibrodysplasia ossificans progressiva.

### BACKGROUND OF THE INVENTION

Some patients with fibrodysplasia ossificans progressiva (FOP) experience muscle, tendon, and/or ligament damage, e.g., soft tissue edema and muscle necrosis, especially concomitant with flare-up symptom onset. These spontaneous muscle, tendon, and/or ligament injuries, and those due to trauma, can lead to heterotopic ossification, can cause tremendous pain, and can incapacitate the affected person. There are no currently approved treatments for muscle, tendon, and/or ligament injury in FOP subjects that prevent heterotopic ossification.

### SUMMARY OF THE INVENTION

The invention features dosing regimens for oral administration of palovarotene and imatinib.

In a first aspect, the invention features a method of treating a subject with fibrodysplasia ossificans progressiva (FOP) characterized by a quiescent period and a non-quiescent period, the method including the steps of: (i) during the quiescent period administering to the subject a therapeutically effective amount of imatinib, or a pharmaceutically acceptable salt thereof, and (ii) during the non-quiescent period administering to the subject a therapeutically effective amount of palovarotene, or a pharmaceutically acceptable salt thereof, wherein during the quiescent period no palovarotene, or a pharmaceutically acceptable salt thereof, is administered to the subject.

In some embodiments, palovarotene or a pharmaceutically acceptable salt thereof is initially administered in a daily loading dose of 20.0±5.0 mg followed by a daily maintenance dose of 10.0±2.5 mg. In some embodiments, the daily loading dose is 20 mg and the daily maintenance dose is 10 mg. In some embodiments, the daily loading dose is 15 mg and the daily maintenance dose is 7.5 mg. In some embodiments, the subject weighs greater than 60 kg.

In some embodiments, palovarotene or a pharmaceutically acceptable salt thereof is initially administered in a daily loading dose of 15.0±2.5 mg followed by a daily maintenance dose of 7.5±2.5 mg. In some embodiments, the daily loading dose is 15 mg and the daily maintenance dose is 7.5 mg. In some embodiments, the daily loading dose is 12.5 mg and the daily maintenance dose is 5 mg. In some embodiments, the subject weighs 40 to 60 kg.

In some embodiments, palovarotene or a pharmaceutically acceptable salt thereof is initially administered in a daily loading dose of 12.5±2.5 mg followed by a daily maintenance dose of 6.0±2.0 mg. In some embodiments, the daily loading dose is 12.5 mg and the daily maintenance dose is 6 mg. In some embodiments, the daily loading dose is 10 mg and the daily maintenance dose is 4 mg. In some embodiments, the subject weighs 20 to 40 kg.

In some embodiments, palovarotene or a pharmaceutically acceptable salt thereof is initially administered in a daily loading dose of 10.0±2.5 mg followed by a daily maintenance dose of 5.0±2.0 mg. In some embodiments, the daily loading dose is 10 mg and the daily maintenance dose is 5 mg. In some embodiments, the daily loading dose is 7.5 mg and the daily maintenance dose is 3 mg. In some embodiments, the subject weighs less than 20 kg.

In some embodiments of any of the above methods, the daily loading dose is administered for a period of 20 to 40 days. In some embodiments, the daily loading does is administered or 28 days. In some embodiments of any of the above methods, the daily maintenance dose is administered for at least a period of 14 to 84 days. In some embodiments, the daily maintenance dose is administered for 56 days.

In some embodiments of any of the above methods, the daily maintenance dose is continued for an additional 28 days if the subject continues to experience flare-ups at the end of 84 days.

In some embodiments of any of the above methods, the subject is an adult.

In some embodiments of any of the above methods, the subject is under 18 years of age and has not achieved 90% skeletal maturity.

In some embodiments of any of the above methods, imatinib is administered daily at a dose of 300 mg to 600 mg (e.g., 300, 350, 400, 450, 500, 550, or 600 mg per day). In some embodiments, imatinib is administered daily at a dose of 400 mg.

In some embodiments of any of the above methods, imatinib is administered daily at a dose of 200 mg/m² to 340 mg/m² (e.g., 200, 210, 220, 230, 240, 250, 260, 280, 290, 300, 310, 320, 330, or 340 mg/m² per day). In some embodiments, imatinib is administered daily at a dose of 340 mg/m².

In another aspect, the invention features a method of treating a subject with FOP characterized by a quiescent period and a non-quiescent period, wherein the subject weighs 60 kg or more, the method including the steps of: (i) during the quiescent period administering daily to the subject 400 mg or 340 mg/m² of imatinib, or a pharmaceutically acceptable salt thereof, and (ii) during the non-quiescent period administering daily to the subject 20 mg of palovarotene, or a pharmaceutically acceptable salt thereof, for 28 days, followed by administering daily to the subject 10 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 10 mg of palovarotene, or a pharmaceutically acceptable salt thereof, is continued for an additional 28 days if the subject continues to experience flare-ups at the end of 84 days

In another aspect, the invention features a method of treating a subject with FOP characterized by a quiescent period and a non-quiescent period, wherein the subject weighs 60 kg or more, the method including the steps of: (i) during the quiescent period administering daily to the subject 400 mg or 340 mg/m² of imatinib, or a pharmaceutically acceptable salt thereof, and (ii) during the non-quiescent period administering daily to the subject 15 mg of palovarotene, or a pharmaceutically acceptable salt thereof, for 28 days, followed by administering daily to the subject 7.5 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 7.5 mg of palovarotene, or a pharmaceutically acceptable salt thereof, is continued for an additional 28 days if the subject continues to experience flare-ups at the end of 84 days.

In another aspect, the invention features a method of treating a subject with FOP characterized by a quiescent period and a non-quiescent period, wherein the subject weighs 40 to 60 kg, the method including the steps of: (i) during the quiescent period administering daily to the subject 400 mg or 340 mg/m² of imatinib, or a pharmaceutically acceptable salt thereof, and (ii) during the non-quiescent period administering daily to the subject 15 mg of palovarotene, or a pharmaceutically acceptable salt thereof, for 28 days, followed by administering daily to the subject 7.5 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 7.5 mg of palovarotene, or a pharmaceutically acceptable salt thereof, is continued for an additional 28 days if the subject continues to experience flare-ups at the end of 84 days.

In another aspect, the invention features a method of treating a subject with FOP characterized by a quiescent period and a non-quiescent period, wherein the subject weighs 40 to 60 kg, the method including the steps of: (i) during the quiescent period administering daily to the subject 400 mg or 340 mg/m² of imatinib, or a pharmaceutically acceptable salt thereof, and (ii) during the non-quiescent period administering daily to the subject 12.5 mg of palovarotene, or a pharmaceutically acceptable salt thereof, for 28 days, followed by administering daily to the subject 5 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 5 mg of palovarotene, or a pharmaceutically acceptable salt thereof, is continued for an additional 28 days if the subject continues to experience flare-ups at the end of 84 days.

In another aspect, the invention features a method of treating a subject with FOP characterized by a quiescent period and a non-quiescent period, wherein the subject weighs 20 to 40 kg, the method including the steps of: (i) during the quiescent period administering daily to the subject 340 mg/m² of imatinib, or a pharmaceutically acceptable salt thereof, and (ii) during the non-quiescent period administering daily to the subject 12.5 mg of palovarotene, or a pharmaceutically acceptable salt thereof, for 28 days, followed by administering daily to the subject 6 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 6 mg of palovarotene, or a pharmaceutically acceptable salt thereof, is continued for an additional 28 days if the subject continues to experience flare-ups at the end of 84 days.

In another aspect, the invention features a method of treating a subject with FOP characterized by a quiescent period and a non-quiescent period, wherein the subject weighs 20 to 40 kg, the method including the steps of: (i) during the quiescent period administering daily to the subject 340 mg/m² of imatinib, or a pharmaceutically acceptable salt thereof, and (ii) during the non-quiescent period administering daily to the subject 10 mg of palovarotene, or a pharmaceutically acceptable salt thereof, for 28 days, followed by administering daily to the subject 4 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 4 mg of palovarotene, or a pharmaceutically acceptable salt thereof, is continued for an additional 28 days if the subject continues to experience flare-ups at the end of 84 days.

In another aspect, the invention features a method of treating a subject with FOP characterized by a quiescent period and a non-quiescent period, wherein the subject weighs less than 20 kg, the method including the steps of: (i) during the quiescent period administering daily to the subject 340 mg/m² of imatinib, or a pharmaceutically acceptable salt thereof, and (ii) during the non-quiescent period administering daily to the subject 10 mg of palovarotene, or a pharmaceutically acceptable salt thereof, for 28 days, followed by administering daily to the subject 5 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 5 mg of palovarotene, or a pharmaceutically acceptable salt thereof, is continued for an additional 28 days if the subject continues to experience flare-ups at the end of 84 days.

In another aspect, the invention features a method of treating a subject with FOP characterized by a quiescent period and a non-quiescent period, wherein the subject weighs less than 20 kg, the method including the steps of: (i) during the quiescent period administering daily to the subject 340 mg/m² of imatinib, or a pharmaceutically acceptable salt thereof, and (ii) during the non-quiescent period administering daily to the subject 7.5 mg of palovarotene, or a pharmaceutically acceptable salt thereof, for 28 days, followed by administering daily to the subject 3 mg of palovarotene, or a pharmaceutically acceptable salt thereof, for 56 days, wherein during the quiescent period no palovarotene, or a pharmaceutically acceptable salt thereof, is administered to the subject. In some embodiments, daily administration of 3 mg of palovarotene, or a pharmaceutically acceptable salt thereof, is continued for an additional 28 days if the subject continues to experience flare-ups at the end of 84 days.

In some embodiments of any of the above methods, the subject is an adult. In some embodiments, the daily dose of imatinib administered during the quiescent period is 400 mg.

In some embodiments of any of the above methods, the subject is under 18 years of age and has not achieved 90% skeletal maturity. In some embodiments, the daily dose of imatinib administered during the quiescent period is 340 mg/m².

In another aspect, the invention features a method of treating a subject under 18 years of age with fibrodysplasia ossificans progressiva (FOP) characterized by a quiescent period and a non-quiescent period, the method including the steps of: (i) providing a subject weighing greater than 60 kg with FOP, (ii) determining the skeletal maturity of the subject, (iii) on the basis of step (ii), if the subject is skeletally immature administering to the subject a daily loading dose of 15.0±1.0 mg followed by a daily maintenance dose of 7.5±1.0 mg of palovarotene, or a pharmaceutically acceptable salt thereof, during the non-quiescent period, or if the subject is skeletally mature administering to the subject a daily loading dose of 20.0±1.0 mg followed by a daily maintenance dose of 10.0±1.0 mg of palovarotene, or a pharmaceutically acceptable salt thereof, during the non-quiescent period. In some embodiments, the daily loading dose for the skeletally immature subject is 15 mg and the daily maintenance dose for the skeletally immature subject is 7.5 mg. In some embodiments, the daily loading dose for the skeletally mature subject is 20 mg and the daily maintenance dose for the skeletally mature subject is 10 mg.

In another aspect, the invention features a method of treating a subject under 18 years of age with fibrodysplasia ossificans progressiva (FOP) characterized by a quiescent period and a non-quiescent period, the method including the steps of: (i) providing a subject weighing 40 to 60 kg with FOP, (ii) determining the skeletal maturity of the subject, (iii) on the basis of step (ii), if the subject is skeletally immature administering to the subject a daily loading dose of 15.0±2.5 mg followed by a daily maintenance dose of 7.5±2.5 mg of palovarotene, or a pharmaceutically acceptable salt thereof, during the non-quiescent period, or if the subject is skeletally mature administering to the subject a daily loading dose of 20.0±1.0 mg followed by a daily maintenance dose of 10.0±1.0 mg of palovarotene, or a pharmaceutically acceptable salt thereof, during the non-quiescent period. In some embodiments, the daily loading dose for the skeletally immature subject is 15 mg and the daily maintenance dose for the skeletally immature subject is 7.5 mg. In some embodiments, the daily loading dose for the skeletally immature subject is 12.5 mg and the daily maintenance dose for the skeletally immature subject is 5 mg. In some embodiments, the daily loading dose for the skeletally mature subject is 20 mg and the daily maintenance dose for the skeletally mature subject is 10 mg.

In another aspect, the invention features a method of treating a subject under 18 years of age with fibrodysplasia ossificans progressiva (FOP) characterized by a quiescent period and a non-quiescent period, the method including the steps of: (i) providing a subject weighing 30 to 40 kg with FOP, (ii) determining the skeletal maturity of the subject, (iii) on the basis of step (ii), if the subject is skeletally immature administering to the subject a daily loading dose of 12.5±2.5 mg followed by a daily maintenance dose of 6.0±2.0 mg of palovarotene, or a pharmaceutically acceptable salt thereof, during the non-quiescent period, or if the subject is skeletally mature administering to the subject a daily loading dose of 20.0±1.0 mg followed by a daily maintenance dose of 10.0±1.0 mg of palovarotene, or a pharmaceutically acceptable salt thereof, during the non-quiescent period. In some embodiments, the daily loading dose for the skeletally immature subject is 12.5 mg and the daily maintenance dose for the skeletally immature subject is 6 mg. In some embodiments, the daily loading dose for the skeletally immature subject is 10 mg and the daily maintenance dose for the skeletally immature subject is 4 mg. In some embodiments, the daily loading dose for the skeletally mature subject is 20 mg and the daily maintenance dose for the skeletally mature subject is 10 mg.

In some embodiments of any of the above methods, the daily loading dose is administered for a period of 20 to 40 days. In some embodiments, the daily loading does is administered or 28 days.

In some embodiments of any of the above methods, the daily maintenance dose is administered for at least a period of 14 to 84 days. In some embodiments, the daily maintenance dose is administered for 56 days.

In some embodiments of any of the above methods, the daily maintenance dose is continued for an additional 28 days if the subject continues to experience flare-ups at the end of 84 days.

In some embodiments of any of the above methods, the method further includes administering to the subject during the quiescent period imatinib, or a pharmaceutically acceptable salt thereof, daily at a dose of 300 mg to 600 mg (e.g., 300, 350, 400, 450, 500, 550, or 600 mg per day). In some embodiments, imatinib is administered daily at a dose of 400 mg.

In some embodiments of any of the above methods, the method further includes administering to the subject during the quiescent period imatinib, or a pharmaceutically acceptable salt thereof, daily at a dose of 200 mg/m² to 340 mg/m² (e.g., 200, 210, 220, 230, 240, 250, 260, 280, 290, 300, 310, 320, 330, or 340 mg/m² per day). In some embodiments, imatinib is administered daily at a dose of 340 mg/m².

In some embodiments of any of the above methods, skeletal maturity is determined using knee and/or hand/wrist radiographs (e.g., to evaluate the status of epiphyseal growth plates).

In some embodiments of any of the above methods, no palovarotene, or a pharmaceutically acceptable salt thereof, is administered to the subject during the quiescent period.

In some embodiments of any of the above methods of treating a subject under 18 years of age, the method further includes administering to the subject a daily dose of 5.0±1.0 mg of palovarotene, or a pharmaceutically acceptable salt thereof during the quiescent period.

In some embodiments of any of the above methods, the subject is between 11 and 17 years old. In some embodiments of any of the above methods, the subject is between 11 and 16 years old. In some embodiments of any of the above methods, imatinib is also administered during the non-quiescent period (e.g., administered daily during the non-quiescent period). In some embodiments, the dose of imatinib administered during the non-quiescent period is the same as the dose administered during the quiescent period.

In some embodiments of any of the above methods, imatinib is not administered during the non-quiescent period.

In some embodiments of any of the above methods, imatinib is administered in an oral liquid formulation.

In some embodiments of any of the above methods, palovarotene is administered in an oral liquid formulation.

In some embodiments of any of the above methods, the method reduces heterotopic ossification in the subject.

In some embodiments of any of the above methods, the method reduces the severity of flare-ups or flare-up symptoms in the subject.

In some embodiments of any of the above methods, the method reduces the flare-up rate in the subject.

### Definitions

As used herein, the term “heterotopic ossification” or “HO” refers to the presence of bone in soft tissue where bone normally does not exist. The HO can be caused by fibrodysplasia ossificans progressiva, a rare genetic condition.

As used herein, the term “reducing the flare-up rate” refers to a reduction in the number or frequency of flare-ups in subjects undergoing treatment with palovarotene and imatinib using the methods described herein in comparison to subjects treated with palovarotene alone or subjects treated with imatinib alone.

As used herein, the term “quiescent period” refers to time periods during which a subject with FOP is not experiencing a non-quiescent period.

As used herein, the term “non-quiescent period” refers to time periods during which a subject with FOP is experiencing a flare-up or is at risk of heterotopic ossification triggered by a flare-up or surgery.

As used herein, the term “reducing the severity of flare-ups” refers to an average reduction in one or more flare-up symptoms in subjects undergoing treatment with palovarotene and imatinib using the methods described herein in comparison to subjects treated with palovarotene alone or subjects treated with imatinib alone.

As used herein, the term “reducing heterotopic ossification” refers to the average reduction in the amount of bone formed, or number of sites at which bone is formed, in soft tissue by subjects undergoing treatment with palovarotene and imatinib using the methods described herein in comparison to subjects treated with palovarotene alone or subjects treated with imatinib alone.

As used herein, the term “flare-up” refers to symptoms related to a local inflammation at an anatomical site where HO is initiated. In FOP subjects a local flare-up is characterized by swelling, pain, erythema, warmth, stiffness and decreased range of motion preceding overt bone formation. Such local inflammation, and early stage lesions, can be associated with the presence and accumulation of innate immune cells, including mast cells that are thought to have an important role in inducing and initiating the HO formation process. As a consequence, the current standard of care for FOP patients includes systemic treatment with corticosteroids within 24 hours of the onset of a flare-up, with treatment continued for several days to reduce inflammation and pain. However, corticosteroids have not been shown to reliably prevent HO. Flare-ups are often injury induced and can include, for example, flare-ups following surgery to excise bone from a soft tissue in FOP subject. The methods described herein can be useful for the treatment of FOP following surgery.

### DETAILED DESCRIPTION OF THE INVENTION

The invention features dosing regimens for the treatment of a subject with fibrodysplasia ossificans progressiva (FOP). FOP is a chronic disease characterized by episodes of acute flare-ups that result in new heterotopic ossification formation and progressively worsening disability. Interspersed with periods of flare-up activity (i.e., non-quiescent periods) are variable-length intervals of apparent disease quiescence in which clinical symptoms are not present. The regimens described herein include administration of palovarotene exclusively during a non-quiescent period (e.g., a period in which the subject is experiencing flare-up activity or is at increased risk of heterotopic ossification due to surgery or flare-up) and administration of imatinib during the quiescent period. The dosing regimens can reduce heterotopic ossification, reduce the number of flare-ups, and/or reduce the severity of flare-ups in subjects suffering from FOP.

### I. Fibrodysplasia Ossificans Progressiva (FOP)

The dosing regimens of palovarotene and imatinib described herein can reduce muscle damage and heterotopic ossification in a subject with FOP. Palovarotene (also called 4-[(1E)-2-[5,6,7,8-Tetrahydro-5,5,8,8-tetramethyl-3-(1H-pyrazol-1-ylmethyl)-2-naphthalenyl]-ethenyl]-benzoic acid) is a retinoic acid receptor gamma (RARγ) selective agonist having the structure:

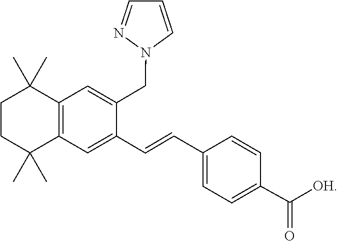 [SMILES: CC1(C)CCC(C)(C)c2cc(Cn3cccn3)c(/C=C/c3ccc(C(=O)O)cc3)cc21]

Imatinib is a 2-phenyl amino pyrimidine derivative that inhibits ABL, c-kit, and PDGF-R, and that is typically used to treat cancer, such as chronic myeloid leukemia.

In some embodiments, palovarotene and imatinib are administered under the dosing regimens described herein to a subject with FOP. FOP is a rare, severely disabling disease characterized by painful, recurrent episodes of soft tissue swelling (flare-ups) and abnormal heterotopic ossification (HO) in muscles, tendons, and ligaments. Flare-up symptoms, their progression and frequency in FOP patients have been described in detail (e.g. Pignolo, R. J. et al. J. Bone Miner. Res. 31, 650-656 (2016)). Mast cell numbers are highly increased in or near FOP lesional tissue, and may potentially contribute to the pathology of FOP.

In some embodiments, lesions begin in early childhood and lead to progressive ankyloses of major joints with resultant loss of movement. Prognosis is poor and median life expectancy is 40 years. FOP is caused by an activating mutation in the bone morphogenetic protein (BMP) type I receptor, or activin receptor type 1A (ACVR1), also known as activin-like-kinase 2 (ALK2) type I receptor. Most patients with FOP have the same point mutation, R206H, termed classical FOP, although roughly 3% of patients have other mutations in the same gene. The prevalence is estimated at approximately 1 in 2 million individuals, with no geographic, ethnic, racial, or gender preference. Heterotopic ossification is episodic and cumulative throughout life, resulting in segments, sheets, and ribbons of extra bone developing throughout the body and across joints, progressively restricting movement. Rapidly growing bony spurs have been known to protrude through the skin causing pain and a risk of infections. Asymmetric HO in the rib cage and subsequent contralateral growth can lead to a rapid progression in spinal deformity and cause thoracic insufficiency syndrome. Ankyloses of the temporomandibular joints results in severe tooth decay and malnutrition.

In some embodiments, periods of flare-up activity are interspersed with variable-length intervals of apparently quiescent disease in the absence of obvious clinical symptoms.

In a patient with FOP, heterotopic ossification (HO) can be measured using a variety of methods known in the art including but not limited to low dose CT-scan imaging, magnetic resonance imaging (MRI), and X-ray imaging. One clinically relevant measure of HO in FOP is the proportion of flare-ups in a patient with no new HO, the number of flare-ups or non-quiescent periods experienced by a patient over a given time-period that do not result in new heterotopic bone formation.

### II. Pharmaceutical Composition and Formulation

For administration to a subject, palovarotene or imatinib can be provided in pharmaceutically acceptable compositions. These pharmaceutically acceptable compositions include palovarotene or imatinib and one or more pharmaceutically acceptable carriers and excipients. Pharmaceutical compositions may be formulated for administration in solid or liquid form.

The palovarotene can be administered in neutral form (i.e., the free base or zwitterionic neutral form). Optionally, palovarotene may be administered as a pharmaceutically acceptable salt, such as a non-toxic acid addition salts or metal complexes that are commonly used in the pharmaceutical industry. Examples of acid addition salts that could be used in the methods of the invention include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes that could be used in the methods of the invention include calcium, zinc, and iron, among others.

The imatinib can be administered in a salt form (e.g., as a mesylate salt).

In some embodiments, a pharmaceutical composition including palovarotene or imatinib is prepared for oral administration. In some embodiments, a pharmaceutical composition is formulated by combining palovarotene or imatinib with one or more pharmaceutically acceptable carriers and excipients. Such carriers and excipients enable the pharmaceutical composition to be formulated as tablets, pills, dragees, capsules, liquids, gels, syrups, slurries, and suspensions, for oral ingestion by a subject.

In some embodiments, pharmaceutical compositions for oral use are obtained by mixing palovarotene or imatinib with one or more carriers and excipients. Suitable carriers and excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In some embodiments, such a mixture is optionally ground and auxiliaries are optionally added. In some embodiments, pharmaceutical compositions are formed to obtain tablets or dragee cores. In some embodiments, disintegrating agents (e.g., cross-linked polyvinyl pyrrolidone, agar, or alginic acid or a salt thereof, such as sodium alginate) are added.

When palovarotene or imatinib is administered orally, a pharmaceutical composition containing palovarotene or imatinib may be in unit dosage form (e.g., liquid or solid unit dosage form). The concentration and/or amount of palovarotene or imatinib in the formulation may vary depending on, e.g., the dosage of palovarotene or imatinib to be administered and the frequency of administration.

### III. Therapy and Dosage

Palovarotene may be administered to a subject with FOP during a flare-up (e.g., acute daily treatment during a non-quiescent period, or for a period following initiation of a non-quiescent period from 84 days to 112 or 140 days in length during which there is risk of further heterotopic ossification). The end of the non-quiescent treatment period is marked by a reduction in the risk of heterotopic ossification, in some cases 84 days following the initiation of non-quiescent period treatment, i.e., 84 days following occurrence of flare-up symptoms. If a subject experiences persistent flare-up symptoms, the non-quiescent treatment period can be extended by, e.g., another 28 days or 56 days (e.g., in four week increments). In some embodiments, treatment during the non-quiescent period involves the use of two different doses of palovarotene: an initial daily dose administered during a first time period (the loading dose), and a lower daily dose administered during a second time period (the maintenance dose). At the end of the non-quiescent period, the administration of palovarotene will cease, and the patient can return to treatment with imatinib. In some embodiments, administration of imatinib continues during the non-quiescent period (e.g., imatinib is administered chronically regardless of whether the patient is in a quiescent or non-quiescent period). The determination about when to transition from non-quiescent dosing to quiescent dosing, i.e. the end of the non-quiescent period, depends upon, and can be defined by, the symptoms experienced by the subject, a determination that can be made in consultation with a subject's care provider or by the patient alone. In the dosing regimens of the invention, the subject can return to a non-quiescent dosing level after the subject has not experienced flare-up symptoms for at least 2 weeks, 3 weeks, 4 weeks, or 5 weeks consecutively.

In some embodiments, 20 mg palovarotene daily is administered for 28 days following initiation of a non-quiescent period, followed by 10 mg palovarotene daily for 56 days, leading to an 84 day treatment period. Optionally treatment with 10 mg palovarotene daily can be extended for an additional 28 days or 56 days (e.g., extended in 4 week increments) if the patient continues to experience flare-up symptoms (i.e., the non-quiescent period persists). Once the patient is no longer experiencing any flare-up symptoms (e.g., at the end of the non-quiescent period) the patient can be treated with imatinib during the quiescent period. Optionally, treatment with imatinib can continue if the patient experiences a flare-up (e.g., palovarotene and imatinib can both be administered during a non-quiescent period).

Palovarotene dosing regimens of the inventions comprise administering a higher dose following initiation of a non-quiescent phase, referred to as a loading dose (e.g., 20 mg/day) for 28 days, followed by a maintenance dose (e.g., 10 mg/day for 56 days). The maintenance dose can be continued in 4 week increments (e.g., for an additional 28 days or 56 days) if, at the end of the 84 day treatment period, the subject continues to experience flare-up symptoms. Adult loading doses for use in the treatment regimens of the invention are from 10 to 20 mg/day and maybe adjusted based on patient weight or palovarotene tolerability in a patient. Adult maintenance doses for use in the treatment regimens of the invention are from 7.5 to 10 mg/day and may be adjusted based on patient weight or palovarotene tolerability in a patient. Exemplary adult dosing regimens are provided in Table 1, below. Adult subjects can also be administered the de-escalated loading and/or maintenance doses shown in Table 2, below.

TABLE 1
Dosing Levels for Adult Patients

| Adult Weight Group | Daily Non- | Daily NQ |
|---|---|---|
| (>18 or <18 with >90% | quiescent (NQ) | Maintenance |
| skeletal maturity) | Loading Dose | Dose |
| Standard Adult Dosing | 20 mg | 10 mg |
| De-escalated | 15 mg | 7.5 mg |
| Adult Dosing 1 | | |
| De-escalated | 12.5 mg | 5 mg |
| Adult Dosing 2 | | |
| De-escalated | 10 mg | 5 mg |
| Adult Dosing 3 | | |

In some embodiments, the dose of palovarotene administered to a child or adolescent (e.g., a subject under 18 years of age) is determined based on whether the subject has reached greater than 90% skeletal maturity. Skeletal maturity can be assessed using knee (anterior/posterior view) and/or hand/wrist radiographs (posterior/anterior view) (e.g., x-rays) to determine whether a subject has open or closed epiphyseal growth plates and to assess the distal femoral angle. Subjects may also undergo standardized stadiometry and knee height for assessments of linear growth, and bilateral hand/wrist and knee growth plate morphology can be assessed by WBCT scan safety read. Bone age can be determined by comparing the hand/wrist radiographs to the atlas standards of Greulich and Pyle. Skeletal maturity can be assessed based on the standards from the Bayley-Pinneau tables (an appendix in the Greulich and Pyle Atlas). These standards can be used to determine whether the subject has reached ≥90% skeletal maturity and whether the subject has reached 100% skeletal maturity. The criteria for achieving 90% of mature height are boys with a hand/wrist bone age of at least 14 years 0 months; and girls with a bone age of at least 12 years 0 months. These bone age criteria correspond with achievement of 91.34+/−2.7% of growth for girls and 95.39+/−1.488% of growth for boys. The cut-off of 90% skeletal maturity is chosen to balance the potential benefit of palovarotene treatment in preventing new heterotopic ossification (HO) with the potential risk of adversely affecting the growth plate, with subsequent effects on linear height. As the majority of growth would have been reached with these values, any potential adverse impact of palovarotene on the growth plate or growth would have minimal impact on overall adult height. The criteria for achieving 100% of mature height are boys with a hand/wrist bone age of at least 18 years 0 months; and girls with a bone age of at least 16 years 0 months.

If a subject is found to have 90% skeletal maturity (e.g., to be skeletally immature), the subject can be administered a weight-adjusted daily dose of palovarotene shown in Table 2, below. For example, a skeletally immature pediatric subject weighing 20 to 40 kg may be administered a daily loading dose of 12.5 mg palovarotene for 28 days, followed by a daily maintenance dose of 6 mg palovarotene for 56 days, which can be continued for an additional 28 or 56 days if the subject continues to experience flare-up symptoms at the end of the initial 84 day treatment period. Subjects found to be skeletally immature can continue knee and hand/wrist radiographs, and linear and knee height measurements. Once a subject has achieved 100% skeletal maturity (confirmed by radiography and defined by growth plate closures), knee and hand/wrist radiographs will no longer be required. In addition, once a subject is 18 years old, linear and knee height growth assessments will no longer be required. If 100% skeletal maturity is not achieved at both locations, then only the location that is still maturing would need to be monitored.

TABLE 2
Weight-Adjusted Dosing Levels for Pediatric
Patients (Under 18 and skeletal maturity ≤90%) and
Dose De-escalation for All Subjects

| | Daily Non- | Daily NQ |
|---|---|---|
| Weight Range | quiescent (NQ) | Maintenance |
| Category | Loading Dose | Dose |
| 10 to <20 kg Regimen | 10 mg | 5 mg |
| 10 to <20 kg De-Escalation | 7.5 mg | 3 mg |
| Regimen | | |
| 20 to <40 kg Regimen | 12.5 mg | 6 mg |
| 20 to <40 kg De-Escalation | 10 mg | 4 mg |
| Regimen | | |
| 40 to <60 kg Regimen | 15 mg | 7.5 mg |
| 40 to <60 kg De-Escalation | 12.5 mg | 5 mg |
| Regimen | | |
| ≥60 kg Regimen | 20 mg | 10 mg |
| ≥60 kg De-Escalation | 15 mg | 7.5 mg |
| Regimen | | |

The dose of imatinib administered during the quiescent period can be a daily dose of about 200 mg/m² to about 340 mg/m² (e.g., 200, 210, 220, 230, 240, 250, 260, 280, 290, 300, 310, 320, 330, or 340 mg/m² per day). In some embodiments, the dose of imatinib is 340 mg/m² per day. The dose of about 200 mg/m² to about 340 mg/m² imatinib per day can be administered to both adult and pediatric subjects. and can be administered in a single dose or in two parts (e.g., two doses of 170 mg/m² can be administered per day to a subject being treated with 340 mg/m² imatinib per day). Adults can also be treated with a dose of about 300 to about 600 mg imatinib per day (e.g., 300, 350, 400, 450, 500, 550, or 600 mg per day). In some embodiments, adult subjects are treated with 400 mg imatinib per day. In some embodiments, the daily dose of imatinib is administered once per day. In some embodiments, the daily dose of imatinib is attained by administering imatinib twice a day (e.g., two doses of 170 mg/m² can be administered per day to a subject being treated with 340 mg/m² imatinib per day, or two doses of 200 mg can be administered per day to an adult subject being treated with 400 mg imatinib per day). Imatinib can be administered in a solid form (e.g., an oral tablet or capsule) or in a liquid form (e.g., an oral liquid formulation). In embodiments in which imatinib is also administered during the non-quiescent period, the dose of imatinib administered to the subject may be the same as the quiescent period dose.

Typically, palovarotene is administered in the form of a dosage unit (e.g., tablet, capsule, etc.). In some embodiments, palovarotene is administered in a dose selected from 1.5 mg, 2 mg, 2.5 mg, 3 mg, 4 mg, 5 mg, 6 mg, 7.5 mg, 10 mg, 12.5 mg, 15 mg, or 20 mg.

In some embodiments, the dose of palovarotene or imatinib can be administered at intervals ranging from more than once per day (e.g., twice per day or three times per day) to once daily, for as long as needed to sustain the desired effect.

### EXAMPLES

### Example 1—Treatment of a Skeletally Mature Subject with FOP

According to the methods disclosed herein, a physician of skill in the art can treat a subject, such as an adult human subject (e.g., a subject having at least 90% skeletal maturity) having FOP, with palovarotene and imatinib to reduce heterotopic ossification, reduce the flare-up rate, or reduce the severity of flare-ups. To this end, a physician of skill in the art can administer to a daily dose of 400 mg of imatinib (e.g., imatinib mesylate) to the subject during a quiescent period (e.g., during a period in which symptoms of a flare-up are not present). Imatinib may be administered in an oral liquid formulation. When the subject begins to experience symptoms of a flare-up (e.g., upon initiation of a non-quiescent period), administration of imatinib is discontinued and the subject can be administered (e.g., orally administered) a daily loading dose of 20 mg palovarotene for 28 days, followed by a daily maintenance dose of 10 mg palovarotene for 56 days. If the subject continues to experience flare-up symptoms at the end of the 84 day treatment period, the daily maintenance dose of 10 mg palovarotene can be extended for 4 week increments (e.g., for 28 days, 56 days, etc.) until the subject no longer experiences flare-up symptoms. At the end of the non-quiescent period, palovarotene administration is discontinued and imatinib administration is resumed at 400 mg per day (e.g., imatinib is administered when the subject is again in a quiescent period).

A practitioner of skill in the art can evaluate the subject's response to the dosage regimen by a variety of methods. For example, a practitioner can measure heterotopic ossification (HO) using low dose CT-scan imaging, magnetic resonance imaging (MRI), or X-ray imaging. A finding that less bone is formed (e.g., the volume of new bone formation is lower) in the subject treated with palovarotene and imatinib compared to a subject treated with imatinib alone or a subject treated with palovarotene alone indicates that the dosage regimen in which imatinib is administered during the quiescent period and palovarotene is administered during the non-quiescent period is more effective than treatment with either agent individually.

### Example 2—Treatment of a Subject with FOP Who has not Achieved 90% Skeletal Maturity

According to the methods disclosed herein, a physician of skill in the art can treat a subject, such as an adolescent human subject having FOP and weighing 40 to 60 kg, with palovarotene and imatinib to reduce heterotopic ossification, reduce the flare-up rate, or reduce the severity of flare-ups. Before initiating treatment, the subject's skeletal maturity can be assessed using knee and/or hand/wrist radiographs. If the subject is found to be skeletally immature (e.g., the subject is found to have 90% skeletal maturity), the dosage regimen can be designed to include a weight-adjusted daily dose of palovarotene (e.g., a dose listed in Table 2). To this end, a physician of skill in the art can administer to a daily dose of 340 mg/m² imatinib (e.g., imatinib mesylate) to the adolescent subject during a quiescent period (e.g., during a period in which symptoms of a flare-up are not present). Imatinib may be administered in an oral liquid formulation. When the subject begins to experience symptoms of a flare-up (e.g., upon initiation of a non-quiescent period), administration of imatinib is discontinued and the subject can be administered (e.g., orally administered) a daily loading dose of 12.5 mg palovarotene for 28 days, followed by a daily maintenance dose of 5 mg palovarotene for 56 days (the weight-adjusted dose for a subject weighing 40 to 60 kg). If the subject continues to experience flare-up symptoms at the end of the 84 day treatment period, the daily maintenance dose of 5 mg palovarotene can be extended for 4 week increments (e.g., for 28 days, 56 days, etc.) until the subject no longer experiences flare-up symptoms. At the end of the non-quiescent period, palovarotene administration is discontinued and imatinib administration is resumed at 340 mg/m² per day (e.g., imatinib is administered when the subject is again in a quiescent period).

A practitioner of skill in the art can evaluate the subject's response to the dosage regimen by a variety of methods. For example, a practitioner can measure heterotopic ossification (HO) using low dose CT-scan imaging, magnetic resonance imaging (MRI), or X-ray imaging. A finding that less bone is formed (e.g., the volume of new bone formation is lower) in the subject treated with palovarotene and imatinib compared to a subject treated with imatinib alone or a subject treated with palovarotene alone indicates that the dosage regimen in which imatinib is administered during the quiescent period and palovarotene is administered during the non-quiescent period is more effective than treatment with either agent individually.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

Other embodiments are within the following claims.

What is claimed is:

1. A method of treating a subject with fibrodysplasia ossificans progressiva (FOP) characterized by a quiescent period and a non-quiescent period, the method comprising:
(i) during the quiescent period administering to the subject a therapeutically effective amount of imatinib, or a pharmaceutically acceptable salt thereof, and
(ii) during the non-quiescent period administering to the subject a therapeutically effective amount of palovarotene, or a pharmaceutically acceptable salt thereof,
wherein during the quiescent period no palovarotene, or a pharmaceutically acceptable salt thereof, is administered to the subject.

2. The method of claim 1, wherein palovarotene or a pharmaceutically acceptable salt thereof is initially administered in a daily loading dose of 20.0±5.0 mg followed by a daily maintenance dose of 10.0±2.5 mg.

3. The method of claim 2, wherein the daily loading dose is 20 mg and the daily maintenance dose is 10 mg.

4. The method of claim 2, wherein the daily loading dose is 15 mg and the daily maintenance dose is 7.5 mg.

5. The method any one of claims 2-4, wherein the subject weighs greater than 60 kg.

6. The method of claim 1, wherein palovarotene or a pharmaceutically acceptable salt thereof is initially administered in a daily loading dose of 15.0±2.5 mg followed by a daily maintenance dose of 7.5±2.5 mg.

7. The method of claim 6, wherein the daily loading dose is 15 mg and the daily maintenance dose is 7.5 mg.

8. The method of claim 6, wherein the daily loading dose is 12.5 mg and the daily maintenance dose is 5 mg.

9. The method of any one of claims 6-8, wherein the subject weighs 40 to 60 kg.

10. The method of claim 1, wherein palovarotene or a pharmaceutically acceptable salt thereof is initially administered in a daily loading dose of 12.5±2.5 mg followed by a daily maintenance dose of 6.0±2.0 mg.

11. The method of claim 10, wherein the daily loading dose is 12.5 mg and the daily maintenance dose is 6 mg.

12. The method of claim 10, wherein the daily loading dose is 10 mg and the daily maintenance dose is 4 mg.

13. The method of any one of claims 10-12, wherein the subject weighs 20 to 40 kg.

14. The method of claim 1, wherein palovarotene or a pharmaceutically acceptable salt thereof is initially administered in a daily loading dose of 10.0±2.5 mg followed by a daily maintenance dose of 5.0±2.0 mg.

15. The method of claim 14, wherein the daily loading dose is 10 mg and the daily maintenance dose is 5 mg.

16. The method of claim 14, wherein the daily loading dose is 7.5 mg and the daily maintenance dose is 3 mg.

17. The method of any one of claims 14-16, wherein the subject weighs less than 20 kg.

18. The method of any one of claims 2-17, wherein the daily loading dose is administered for a period of 20 to 40 days.

19. The method of claim 18, wherein the daily loading does is administered or 28 days.

20. The method of any one of claims 2-19, wherein the daily maintenance dose is administered for at least a period of 14 to 84 days.

21. The method of claim 20, wherein the daily maintenance dose is administered for 56 days.

22. The method of any one of claims 2-21, wherein the daily maintenance dose is continued for an additional 28 days if the subject continues to experience flare-ups at the end of 84 days.

23. The method of any one of claims 1-22, wherein the subject is an adult.

24. The method of any one of claims 1-22, wherein the subject is under 18 years of age and has not achieved 90% skeletal maturity.

25. The method of any one of claims 1-23, wherein imatinib is administered daily at a dose of 300 mg to 600 mg.

26. The method of claim 25, wherein imatinib is administered daily at a dose of 400 mg.

27. The method of any one of claims 1-24, wherein imatinib is administered daily at a dose of 200 mg/m² to 340 mg/m².

28. The method of claim 27, wherein imatinib is administered daily at a dose of 340 mg/m².

29. A method of treating a subject with FOP characterized by a quiescent period and a non-quiescent period, wherein the subject weighs 60 kg or more, the method comprising:
(i) during the quiescent period administering daily to the subject 400 mg or 340 mg/m² of imatinib, or a pharmaceutically acceptable salt thereof, and
(ii) during the non-quiescent period administering daily to the subject 20 mg of palovarotene, or a pharmaceutically acceptable salt thereof, for 28 days, followed by administering daily to the subject 10 mg of palovarotene, or a pharmaceutically acceptable salt thereof, for 56 days,
wherein during the quiescent period no palovarotene, or a pharmaceutically acceptable salt thereof, is administered to the subject.

30. A method of treating a subject with FOP characterized by a quiescent period and a non-quiescent period, wherein the subject weighs 60 kg or more, the method comprising:
(i) during the quiescent period administering daily to the subject 400 mg or 340 mg/m² of imatinib, or a pharmaceutically acceptable salt thereof, and
(ii) during the non-quiescent period administering daily to the subject 15 mg of palovarotene, or a pharmaceutically acceptable salt thereof, for 28 days, followed by administering daily to the subject 7.5 mg of palovarotene, or a pharmaceutically acceptable salt thereof, for 56 days,
wherein during the quiescent period no palovarotene, or a pharmaceutically acceptable salt thereof, is administered to the subject.

31. A method of treating a subject with FOP characterized by a quiescent period and a non-quiescent period, wherein the subject weighs 40 to 60 kg, the method comprising:
(i) during the quiescent period administering daily to the subject 400 mg or 340 mg/m² of imatinib, or a pharmaceutically acceptable salt thereof, and
(ii) during the non-quiescent period administering daily to the subject 15 mg of palovarotene, or a pharmaceutically acceptable salt thereof, for 28 days, followed by administering daily to the subject 7.5 mg of palovarotene, or a pharmaceutically acceptable salt thereof, for 56 days,
wherein during the quiescent period no palovarotene, or a pharmaceutically acceptable salt thereof, is administered to the subject.

32. A method of treating a subject with FOP characterized by a quiescent period and a non-quiescent period, wherein the subject weighs 40 to 60 kg, the method comprising:
(i) during the quiescent period administering daily to the subject 400 mg or 340 mg/m² of imatinib, or a pharmaceutically acceptable salt thereof, and
(ii) during the non-quiescent period administering daily to the subject 12.5 mg of palovarotene, or a pharmaceutically acceptable salt thereof, for 28 days, followed by administering daily to the subject 5 mg of palovarotene, or a pharmaceutically acceptable salt thereof, for 56 days,
wherein during the quiescent period no palovarotene, or a pharmaceutically acceptable salt thereof, is administered to the subject.

33. A method of treating a subject with FOP characterized by a quiescent period and a non-quiescent period, wherein the subject weighs 20 to 40 kg, the method comprising:
(i) during the quiescent period administering daily to the subject 340 mg/m² of imatinib, or a pharmaceutically acceptable salt thereof, and
(ii) during the non-quiescent period administering daily to the subject 12.5 mg of palovarotene, or a pharmaceutically acceptable salt thereof, for 28 days, followed by administering daily to the subject 6 mg of palovarotene, or a pharmaceutically acceptable salt thereof, for 56 days,
wherein during the quiescent period no palovarotene, or a pharmaceutically acceptable salt thereof, is administered to the subject.

34. A method of treating a subject with FOP characterized by a quiescent period and a non-quiescent period, wherein the subject weighs 20 to 40 kg, the method comprising:
(i) during the quiescent period administering daily to the subject 340 mg/m² of imatinib, or a pharmaceutically acceptable salt thereof, and
(ii) during the non-quiescent period administering daily to the subject 10 mg of palovarotene, or a pharmaceutically acceptable salt thereof, for 28 days, followed by administering daily to the subject 4 mg of palovarotene, or a pharmaceutically acceptable salt thereof, for 56 days,
wherein during the quiescent period no palovarotene, or a pharmaceutically acceptable salt thereof, is administered to the subject.

35. A method of treating a subject with FOP characterized by a quiescent period and a non-quiescent period, wherein the subject weighs less than 20 kg, the method comprising:
(i) during the quiescent period administering daily to the subject 400 mg or 340 mg/m² of imatinib, or a pharmaceutically acceptable salt thereof, and
(ii) during the non-quiescent period administering daily to the subject 10 mg of palovarotene, or a pharmaceutically acceptable salt thereof, for 28 days, followed by administering daily to the subject 5 mg of palovarotene, or a pharmaceutically acceptable salt thereof, for 56 days,
wherein during the quiescent period no palovarotene, or a pharmaceutically acceptable salt thereof, is administered to the subject.

36. A method of treating a subject with FOP characterized by a quiescent period and a non-quiescent period, wherein the subject weighs less than 20 kg, the method comprising:
(i) during the quiescent period administering daily to the subject 400 mg or 340 mg/m² of imatinib, or a pharmaceutically acceptable salt thereof, and
(ii) during the non-quiescent period administering daily to the subject 7.5 mg of palovarotene, or a pharmaceutically acceptable salt thereof, for 28 days, followed by administering daily to the subject 3 mg of palovarotene, or a pharmaceutically acceptable salt thereof, for 56 days,
wherein during the quiescent period no palovarotene, or a pharmaceutically acceptable salt thereof, is administered to the subject.

37. The method of any one of claims 29-36, wherein the subject is an adult.

38. The method of claim 37, wherein the daily dose of imatinib administered during the quiescent period is 400 mg.

39. The method of any one of claims 29-36, wherein the subject is under 18 years of age and has not achieved 90% skeletal maturity

40. The method of claim 39, wherein the daily dose of imatinib administered during the quiescent period is 340 mg/m².

41. A method of treating a subject under 18 years of age with fibrodysplasia ossificans progressiva (FOP) characterized by a quiescent period and a non-quiescent period, the method comprising:
(i) providing a subject weighing greater than 60 kg with FOP,
(ii) determining the skeletal maturity of the subject,
(iii) on the basis of step (ii), if the subject is skeletally immature administering to the subject a daily loading dose of 15.0±1.0 mg followed by a daily maintenance dose of 7.5±1.0 mg of palovarotene, or a pharmaceutically acceptable salt thereof, during the non-quiescent period, or if the subject is skeletally mature administering to the subject a daily loading dose of 20.0±1.0 mg followed by a daily maintenance dose of 10.0±1.0 mg of palovarotene, or a pharmaceutically acceptable salt thereof, during the non-quiescent period.

42. The method of claim 41, wherein the daily loading dose for the skeletally mature subject is 20 mg and the daily maintenance dose for the skeletally mature subject is 10 mg.

43. The method of claim 41, wherein the daily loading dose for the skeletally immature subject is 15 mg and the daily maintenance dose for the skeletally immature subject is 7.5 mg.

44. A method of treating a subject under 18 years of age with fibrodysplasia ossificans progressiva (FOP) characterized by a quiescent period and a non-quiescent period, the method comprising:
(i) providing a subject weighing 40 to 60 kg with FOP,
(ii) determining the skeletal maturity of the subject,
(iii) on the basis of step (ii), if the subject is skeletally immature administering to the subject a daily loading dose of 15.0±2.5 mg followed by a daily maintenance dose of 7.5±2.5 mg of palovarotene, or a pharmaceutically acceptable salt thereof, during the non-quiescent period, or if the subject is skeletally mature administering to the subject a daily loading dose of 20.0±1.0 mg followed by a daily maintenance dose of 10.0±1.0 mg of palovarotene, or a pharmaceutically acceptable salt thereof, during the non-quiescent period.

45. The method of claim 44, wherein the daily loading dose for the skeletally immature subject is 15 mg and the daily maintenance dose for the skeletally immature subject is 7.5 mg.

46. The method of claim 44, wherein the daily loading dose for the skeletally immature subject is 12.5 mg and the daily maintenance dose for the skeletally immature subject is 5 mg.

47. The method of claim 44, wherein the daily loading dose for the skeletally mature subject is 20 mg and the daily maintenance dose for the skeletally mature subject is 10 mg.

48. A method of treating a subject under 18 years of age with fibrodysplasia ossificans progressiva (FOP) characterized by a quiescent period and a non-quiescent period, the method comprising:
(i) providing a subject weighing 30 to 40 kg with FOP,
(ii) determining the skeletal maturity of the subject,
(iii) on the basis of step (ii), if the subject is skeletally immature administering to the subject a daily loading dose of 12.5±2.5 mg followed by a daily maintenance dose of 6.0±2.0 mg of palovarotene, or a pharmaceutically acceptable salt thereof, during the non-quiescent period, or if the subject is skeletally mature administering to the subject a daily loading dose of 20.0±1.0 mg followed by a daily maintenance dose of 10.0±1.0 mg of palovarotene, or a pharmaceutically acceptable salt thereof, during the non-quiescent period.

49. The method of claim 48, wherein the daily loading dose for the skeletally immature subject is 12.5 mg and the daily maintenance dose for the skeletally immature subject is 6 mg.

50. The method of claim 48, wherein the daily loading dose for the skeletally immature subject is 10 mg and the daily maintenance dose for the skeletally immature subject is 4 mg.

51. The method of claim 48, wherein the daily loading dose for the skeletally mature subject is 20 mg and the daily maintenance dose for the skeletally mature subject is 10 mg.

52. The method of any one of claims 41-51, wherein the daily loading dose is administered for a period of 20 to 40 days.

53. The method of claim 52, wherein the daily loading dose is administered or 28 days.

54. The method of any one of claims 41-53, wherein the daily maintenance dose is administered for at least a period of 14 to 84 days.

55. The method of claim 54, wherein the daily maintenance dose is administered for 56 days.

56. The method of any one of claims 41-55, wherein the daily maintenance dose is continued for an additional 28 days if the subject continues to experience flare-ups at the end of 84 days.

57. The method of any one of claims 41-56, further comprising administering to the subject during the quiescent period imatinib, or a pharmaceutically acceptable salt thereof, daily at a dose of 300 mg to 600 mg.

58. The method of claim 57, wherein imatinib is administered daily at a dose of 400 mg.

59. The method of any one of claims 41-56, further comprising administering to the subject during the quiescent period imatinib, or a pharmaceutically acceptable salt thereof, daily at a dose of 200 mg/m² to 340 mg/m².

60. The method of claim 59 wherein imatinib is administered daily at a dose of 340 mg/m².

61. The method of any one of claims 41-60, wherein skeletal maturity is determined using knee and/or hand/wrist radiographs.

62. The method of any one of claims 41-61, wherein during the quiescent period no palovarotene, or a pharmaceutically acceptable salt thereof, is administered to the subject.

63. The method of any one of claims 41-61, further comprising during the quiescent period administering to the subject a daily dose of 5.0±1.0 mg of palovarotene, or a pharmaceutically acceptable salt thereof.

64. The method of any one of claims 41-63, wherein the subject is between 11 and 17 years old.

65. The method of any one of claims 41-63, wherein the subject is between 11 and 16 years old.

66. The method of any one of claims 1-65, wherein imatinib is also administered during the non-quiescent period.

67. The method of claim 66, wherein the dose of imatinib administered during the non-quiescent period is the same as the dose administered during the quiescent period.

68. The method of any one of claims 1-65, wherein imatinib is not administered during the non-quiescent period.

69. The method of any one of claims 1-68, wherein imatinib is administered in an oral liquid formulation.

70. The method of any one of claims 1-69, wherein palovarotene is administered in an oral liquid formulation.

71. The method of any one of claims 1-70, wherein the method reduces heterotopic ossification in the subject.

72. The method of any one of claims 1-71, wherein the method reduces the severity of flare-ups or flare-up symptoms in the subject.

73. The method of any one of claims 1-72, wherein the method reduces the flare-up rate in the subject.
